(19) United States
(12) Patent Application Publication
(10) Pub. No.: US 2024/0315546 A1
(43) Pub. Date: Sep. 26, 2024

(54) SYSTEM FOR DENTAL ASSESSMENT

(71) Applicant: Get-Grin Inc., Airmont, NY (US)

(72) Inventors: Yarden EILAT-BLOCH, Haifa (IL); Carmi RAZ, Gizo (IL)

(21) Appl. No.: 18/679,193

(22) Filed: May 30, 2024

Related U.S. Application Data
(63) Continuation of application No. PCT/US22/51542, filed on Dec. 1, 2022.
(60) Provisional application No. 63/284,683, filed on Dec. 1, 2021.

(51) Int. Cl. A61B 1/247; A61B 1/04; A61B 1/06

(52) U.S. Cl. CPC A61B 1/247; A61B 1/042; A61B 1/0655

(57) ABSTRACT

Described herein are systems and methods for dental assessment, including a dental assessment device, comprising: a body comprising (i) a proximal opening, wherein said proximal opening is configured to be inserted within an intraoral region of a subject, and wherein said proximal opening has dimensions at least equal to that of a surface of a single tooth of said subject; (ii) a distal opening, wherein said body is configured to enable viewing through said proximal opening via said distal opening, and wherein a distance between said proximal opening and said distal opening is at least 60 mm; and (iii) at least one light source configured to provide uniform illumination through said proximal opening at a wavelength in a range of 360 nm to 440 nm.

### CROSS REFERENCE TO RELATED APPLICATIONS

This application is a Continuation Application of International Patent Application PCT/US22/51542 filed Dec. 1, 2022, which claims benefit of U.S. Provisional Application No. 63/284,683 filed Dec. 1, 2021, each of which is incorporated herein by reference in its entirety.

### TECHNICAL FIELD

The systems and methods described herein, in at least some embodiments, relates to devices and methods for dental assessment, and more specifically to devices and methods for performing dental assessment by any person, including a non-dental professional.

### BACKGROUND

Dental professionals may treat and monitor a patient's dental condition based on in-person visits. Treatment and monitoring of a patient's dental condition may require a patient to schedule multiple in-person visits to a dentist. The quality of treatment and the accuracy of monitoring may vary depending on how often and how consistently a patient sees a dentist. In some cases, suboptimal treatment outcomes may result if a patient is unable or unwilling to schedule regular visits to a dentist.

### SUMMARY

Recognized herein is a need for dental monitoring solutions to allow dental assessment, optionally remote dental assessment, without requiring a dental professional to be physically present with the patient. Some dental professionals may use conventional teledentistry solutions to accommodate patients' needs and schedules. However, such conventional teledentistry solutions may provide inadequate levels of supervision. Further, such conventional teledentistry solutions may be limited by an inaccurate or insufficient monitoring of a patient's dental condition based on one or more photos taken by the patient, if the photos do not adequately capture various intraoral features.

The present disclosure provides devices and methods for dental assessment, such as remote dental assessment. As used herein, the term “remote dental assessment” may refer to assessments conducted by remote personnel and may refer to the acquisition of one or more intraoral videos and/or intraoral images that can be assessed using a remote computing device, or can be sent to different remote locations for assessment.

The devices and methods disclosed herein may provide a convenient solution and user experience for dental assessment, in some embodiments, remote assessment, optionally enabling patients to capture one or more intraoral videos or images using a mobile device such as a smartphone. The methods and devices disclosed herein, according to some embodiments, may provide dentists with a detailed analysis of the patient's dental condition based on one or more full arch scans captured remotely by the patient.

In an aspect, provided herein is a dental assessment device, comprising: a body comprising (i) a proximal opening, wherein the proximal opening is configured to be inserted within an intraoral region of a subject, and wherein the proximal opening has dimensions at least equal to that of a surface of a single tooth of the subject: (ii) a distal opening, wherein the body is configured to enable viewing through the proximal opening via the distal opening, and wherein a distance between the proximal opening and the distal opening is at least 60 mm; and (ii) at least one light source configured to provide uniform illumination through the proximal opening at a wavelength in a range of 360 nm to 440 nm.

In some cases, the body is configured to enable viewing through the proximal opening through a camera, and a lens of the camera is positioned to acquire an image or a video through the proximal opening via the distal opening.

In some cases, the camera is a camera of a mobile phone.

In some cases, the body is configured to releasably attach to the camera such that the camera is positioned to acquire an image or a video through the proximal opening via the distal opening.

In some cases, the proximal opening is configured to fit against a human gingiva. In some cases, the proximal opening is curved. In some cases, the proximal end of the body is configured to couple the body to the subject's mouth. In some cases, the proximal end comprises alternating concave and convex regions. In some cases, the concave regions are configured to contact a top and bottom lip of the subject's mouth. In some cases, the concave regions are configured to hold a lip of the subject outside of the proximal end. In some cases, the convex regions are configured to contact a side of the subject's mouth. In some cases, the convex regions are configured to hold a cheek of the subject outside of the proximal end.

In some cases, the dental assessment device further comprises an outwardly extending rim around the proximal opening.

In some cases, a size of the distal opening is configured to enable the distal opening to fit against an outer surface of a human eye.

In some cases, the distal opening has a rectilinear shape.

In some cases, the proximal opening is configured to support an inner surface of a lip of a subject when inserted into an intraoral region of the subject proximal to a gingiva.

In some cases, the body is configured to block substantially all external illumination when inserted into an intraoral region of a subject proximal to a gingiva of the subject.

In some cases, the at least one light source comprises a light strip.

In some cases, the at least one light source is a Light-emitting diode (LED).

In some cases, the dental assessment device further comprises a color reference target.

In some cases, the dental assessment is configured to receive power from an additional device.

In another aspect, provided herein is a method for dental assessment, comprising: providing a dental assessment device: inserting the proximal opening into an intraoral region of a subject: illuminating at least one tooth within the intraoral region of the subject with the at least one light source; and observing a dental fluorescence resulting from the illuminating of the at least one tooth.

In some cases, the method further comprises: providing a camera; and attaching the camera to the device, wherein a lens of the camera is positioned to acquire an image or a video through the proximal opening via the distal opening, wherein the camera is used for the observing of the dental fluorescence.

In some cases, the camera is a camera of a mobile phone. In some cases, the mobile phone comprises software and/or hardware configured to analyze the acquired image or video and to identify a location and/or condition of at least a portion of the at least one tooth of the subject.

In some cases, the condition comprises a change in enamel and/or dentin of the at least one tooth.

In another aspect, provided herein is a band for dental assessment, comprising: an inner face: an outer face: two edges: at least one light source configured to provide illumination at a wavelength in the range of 360 nm to 440 nm, wherein the illumination extends outwardly from at least one of the two edges: a power source configured to provide power to the light source; and a switch configured to activate the light source.

In some cases, the band is configured to be insertable in a dental assessment device, and the dental assessment device comprises (i) a proximal opening configured for insertion within an intraoral region of a subject and (ii) a distal opening.

In some cases, the at least one light source is configured to provide uniform illumination of an intraoral cavity of a subject.

In another aspect, provided herein is a method for dental assessment, comprising: providing a dental assessment device comprising (i) a proximal opening configured for insertion within an intraoral region of a subject, and (ii) a distal opening: providing a band: inserting the band in the dental assessment device, wherein the light source is directed towards the proximal opening of the device: inserting the proximal opening of the device in an intraoral region of a subject: illuminating at least one tooth of the subject within the intraoral region with the light source; and observing a dental fluorescence resulting from the illuminating of the at least one tooth of the subject.

In some cases, the method further comprises providing a camera; and attaching the camera to the dental assessment device, wherein a lens of the camera is positioned to acquire an image or a video through the proximal opening via the distal opening, wherein the camera is used for the observing of the dental fluorescence.

In some cases, the camera is a camera of a mobile phone. In some cases, the mobile phone comprises software or hardware configured to analyze the acquired image or video and to identify a location and/or condition of at least a portion of the at least one tooth of the subject.

In some cases, the condition comprises a change in enamel and/or dentin of the at least one tooth of the subject.

In another aspect, provided herein is an intraoral element comprising: at least one light source: a power source configured to provide power to the light source: a switch configured to activate the light source; and at least one attachment component for releasably coupling the light source to a location within an intraoral region of a subject.

In some cases, the light source is configured to provide illumination at a wavelength in the range of 360 nm to 440 nm.

In some cases, the attachment component comprises a strip configured to be held between upper and lower teeth of the subject.

In some cases, light is emitted from a portion of the light source.

In some cases, the light source provides uniform illumination of the intraoral region of the subject.

In some cases, the switch is configured to be remotely activated.

In some cases, the at least one light source comprises a first light source and a second light source, and the second light source is configured to provide light at a different wavelength range from that of the first light source, and wherein the first light source and the second light source are configured to be activated independently.

In another aspect, provided herein is a method for dental assessment, comprising: providing an intraoral element: inserting the intraoral element into an intraoral region of a subject: using the intraoral element, illuminating an interior surface of at least one tooth of the subject within the intraoral region; and observing a dental fluorescence resulting from the illuminating of the at least one tooth of the subject.

In some cases, the method further comprises: providing a dental assessment device comprising (i) a proximal opening configured for insertion in the intraoral region of the subject and (ii) a distal opening, wherein the observing of the dental fluorescence resulting from the illuminating is performed via the dental assessment device.

In some cases, the method further comprises: providing a camera; and coupling the camera to the dental assessment device, wherein a lens of the camera is positioned to acquire an image or video through the proximal opening via the distal opening, wherein the camera is used for the observing of the dental fluorescence.

In some cases, the camera is a camera of a mobile phone. In some cases, the mobile phone comprises software and/or hardware configured to analyze an acquired dental image or video and to identify a location and/or condition of the at least one tooth of the subject.

In some cases, the condition comprises a change in enamel and/or dentin of the at least one tooth of the subject.

According to some embodiments, the camera and/or the mobile phone comprises software and/or hardware configured to analyze an acquired dental image and to identify a location and/or condition of at least one tooth of the subject. According to some such embodiments, the condition comprises a change (such as a reduction) in enamel and/or dentin. Unless otherwise defined, all technical and scientific terms used herein have the same meaning as commonly understood by one of ordinary skill in the art to which the systems and methods described herein belong. Although suitable methods and materials are described below; methods and materials similar or equivalent to those described herein can be used in the practice of the systems and methods described herein. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Implementation of the method and system of the systems and methods described herein involve performing or completing selected tasks or steps manually, automatically, or a combination thereof. Moreover, according to actual instrumentation and equipment of preferred embodiments of methods, apparatus and systems described herein, several selected steps could be implemented by hardware or by software on any operating system of any firmware or a combination thereof. For example, as hardware, selected steps could be implemented as a chip or a circuit. As software, selected steps could be implemented as a plurality of software instructions being executed by a computer using any suitable operating system. In any case, selected steps of the methods and systems described herein could be described as being performed by a data processor, such as a computing platform for executing a plurality of instructions.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

In order to understand the methods and systems described herein and to see how they may be carried out in practice, embodiments will now be described, by way of non-limiting examples only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIGS. 1A, 1B, and 1C schematically illustrate an example of a dental assessment device providing uniform illumination of an intraoral region of a subject, in accordance with some embodiments.

FIG. 2A schematically illustrates an example of a band, in accordance with some embodiments.

FIGS. 2B and 2C schematically illustrate an example of a band provided within a dental assessment device, in accordance with some embodiments.

FIG. 3A schematically illustrates an example of an intraoral element comprising a light source, in accordance with some embodiments.

FIGS. 3B and 3C schematically illustrate positioning of an intraoral element according to FIG. 3A within an intraoral cavity of a subject, in accordance with some embodiments.

FIG. 4 schematically illustrates a computer system that is programmed or otherwise configured to implement at least some of the methods disclosed herein, in accordance with some embodiments.

FIG. 5 is a flow-chart schematically illustrating methods for dental assessment, in accordance with some embodiments.

FIG. 6 is a flow-chart schematically illustrating methods for dental assessment, in accordance with some embodiments.

FIG. 7 is a flow-chart schematically illustrating methods for dental assessment, in accordance with some embodiments.

### DETAILED DESCRIPTION OF EMBODIMENTS

While various embodiments have been shown and described herein, it will be obvious to those skilled in the art that such embodiments are provided by way of example only. Numerous variations, changes, and substitutions may occur to those skilled in the art without departing from the systems and methods described herein. It should be understood that various alternatives to the embodiments described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

As used herein, when a numerical value is preceded by the term “about”, the term “about” is intended to indicate +/−10% of that value. Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “a,” “an,” and “the,” as used herein, generally refer to singular and plural references unless the context clearly dictates otherwise.

### Overview

In an aspect, the present disclosure provides devices and methods for dental assessment. As used herein, the term “dental assessment” may refer to assessment of at least one tooth or a subject and/or of a condition of at least one tooth of a subject by the naked eye or by image analysis such as assessment of age-related damage, splitting, breakage, deformation, perusing, cavities, loss of teeth or any other pathological issues.

The methods and devices disclosed herein, in at least some embodiments, may provide a convenient solution and user experience for dental patients to capture one or more intraoral videos or images using a mobile device such as a smartphone without the requirement for the physical presence of a dental professional. The methods and devices disclosed herein may provide dentists and orthodontists with a detailed analysis of the patient's dental condition based on at least one image of a tooth and/or full arch scan captured remotely by a user, who may be a non-dental professional, such as the patient.

Fluorescence is an emission of light (photons) by a substance that has absorbed light of higher energy. When exposed to light sources containing ultraviolet components, fluorescence emission with a peak of 440 nm is observed in human teeth. Uniform illumination of at least some of the teeth or of a single tooth of a subject with a specific wavelength of light can result in uniform fluorescence from the illuminated teeth or tooth when the enamel and the dentin are intact. A variation in dental fluorescence can therefore be indicative of irregularities, such as cracking, thinning etc. of the enamel of a tooth or part of a tooth. In an aspect, the present disclosure provides devices to provide equal and constant exposure of light to dental tissue and methods of use thereof.

The present disclosure is of devices and methods for use in dental assessment. The systems and devices of the present disclosure may be implemented using a software application that is configured to enable a user to capture videos and/or images of intraoral regions. The software application may be used by a user or a subject (e.g., a dental patient) in conjunction with a mobile device to remotely monitor a dental condition of the subject. The dental condition may comprise a development of a condition of a tooth, such as a crack or a stain, a growth, a movement, a change in physical appearance, a physical arrangement, a position, and/or an orientation of at least one tooth of a subject.

The dental assessment may be used to enable remote dental monitoring. As used herein, the term “remote monitoring” may refer to monitoring a dental condition of a patient that is performed at one or more locations remote from the patient. For example, a dentist or a medical specialist may monitor the dental anatomy or dental condition at a first location that is different from a second location where the patient is located. The first location and the second location may be separated by a distance spanning at least 1 meter, 1 kilometer, 10 kilometers, 100 kilometers, 1000 kilometers, or more.

The remote monitoring may be performed by assessing a dental condition of the subject using one or more dental scans captured by a user when the patient is located remotely from the dentist or a dental office. In some cases, the remote monitoring may be performed in real-time such that a dentist is able to assess the dental condition when a subject uses a mobile device to acquire one or more dental scan of one or more intraoral regions in the patient's mouth. The remote monitoring may be performed using equipment, hardware, and/or software that is not physically located at a dental clinic.

The software application for dental assessment may be configured to run on a mobile device. The mobile device may comprise a smartphone, a tablet, a laptop, or any suitable device that may be used by a patient to capture one or more dental assessments. The software application may be installed on a mobile device of a patient undergoing a dental treatment or who will be undergoing a dental treatment. The software application may be a patient-side software application. Alternatively, the software application for dental assessment may be configured to run on a fixed-location device, such as a desktop computer.

In some cases, the patient-side software application may be used in a compatible manner with a practitioner-side software application that is accessible by a caregiver. The patient-side software application and the practitioner-side software application may enable real-time communication and sharing of dental assessment, or data between one or more patients and one or more caregivers. The one or more caregivers may comprise, for example, a dentist, an orthodontist, an oral surgeon, individuals having one or more dental specialties, or a dental staff practitioner.

### Device for Dental Assessment

In an aspect, provided herein is a dental assessment device, comprising: a body having at least a proximal opening, configured for insertion within an intraoral region of a subject, the proximal opening having dimensions at least equal to that of a surface of a single tooth and a distal opening, the body configured to enable viewing through the proximal opening via the distal opening, wherein a distance between the proximal opening and the distal opening is at least 60 mm; and at least one light source providing uniform illumination through the proximal opening at a wavelength in a range of 360 nm to 440 nm.

According to some embodiments, a length of the proximal opening is less than about 100 mm, less than about 95 mm, less than about 90 mm, less than about 85 mm, less than about 80 mm, less than about the surface of two teeth.

According to some embodiments, the body is configured to enable viewing using the naked eye.

According to some embodiments, the body is configured to enable viewing through a camera when a lens of the camera is positioned to acquire on image or a video through the proximal opening via the distal opening.

According to some embodiments, the dental assessment device further comprises a camera. According to some embodiments, the camera is any digital camera suitable for use with the dental assessment device, such as a camera of a mobile phone. According to some embodiments, the body of the dental assessment device is configured for reversibly attaching a camera thereto, such that the camera is positioned to acquire an image through the proximal opening via the distal opening. According to some embodiments, the camera is configured to store the image, such as to an electronic storage device, for example a memory card of a digital camera.

According to some embodiments, the proximal opening is curved and configured to fit against a human gingiva, in some embodiments against an upper and lower human gingiva.

According to some embodiments, the proximal opening comprises an outwardly extending rim, in some embodiments of width of some millimeters, in some embodiments of width less than 5 mm, 4 mm or 3 mm. In some embodiments, the rim is configured to support an inner surface of an upper and/or lower lip of a subject when inserted into the intraoral region of the subject, thereby keeping the lips of the subject open around the proximal opening to assist in viewing of the intraoral region.

According to some embodiments, a size of the distal opening is configured to enable said distal opening to fit against an outer surface of a human eye, such as against an outer surface of the eyelids at the rim of the eye.

According to some embodiments, the distal opening has a rectilinear shape, such as a square or rectangular shape.

According to some embodiments, the dental assessment device is configured to block substantially all external illumination when inserted into an intraoral region of a subject proximal to a gingiva i.e. is opaque.

According to some embodiments, the at least one light source comprises a light strip.

According to some embodiments, the at least one light source is a light-emitting diode (LED).

According to some embodiments, the dental assessment device further comprises a color reference target.

According to some embodiments, the dental assessment device is configured to receive power from an additional device such as a fixed or portable external power source, for example, a battery.

According to some embodiments, the configured to receive power comprises a switch. According to some embodiments, the configuration includes wireless connection.

In another aspect, provided herein is a method for dental assessment, comprising: providing a dental assessment device as disclosed herein:
inserting the proximal opening of the device into an intraoral region of a subject: illuminating at least one tooth within the intraoral region of the subject with the at least one light source; and
observing a dental fluorescence resulting from the illuminating of the at least one tooth.

According to some embodiments, the observing of dental fluorescence is carried out using a naked eye.

According to some embodiments, the observing of dental fluorescence is carried out using a camera.

According to some embodiments, the method further comprises providing a camera: attaching the camera to the device wherein a lens of the camera is positioned to acquire an image or a video through the proximal opening via the distal opening. According to some such embodiments, the camera is a camera of a mobile phone.

According to some embodiments, the attaching of the camera to the device can comprises magnets, Velcro or stickers

According to some such embodiments, the mobile phone comprises software and/or hardware configured to analyze the acquired image and to identify a location and/or condition of at least a portion of at least one tooth of a subject. According to some such embodiments, the location and/or condition is of more than one tooth, in some embodiments of all of the teeth of the subject.

According to some embodiments, a condition of a tooth is a change, such as a reduction of amount or density, of enamel and/or dentin.

In another aspect, provided herein is a band having an inner face, an outer face and two edges, the band comprising at least one light source providing illumination at a wavelength in the range of 360 nm to 440 nm: a power source configured to provide power to the light source; and a switch configured to activate the light source, wherein the light source provides illumination extending outwardly from one of the two edges.

According to some embodiments, the band is a self-expanding band, i.e. a band which can be provided in a compact form when restrained, which expands upon removal of a restraint.

According to some embodiments, the band is configured to be insertable within a dental assessment device comprising at least a proximal opening configured for insertion within an intraoral region of a subject and a distal opening. In some such embodiments, the band is configured to expand until coming into contact with an inner surface of the dental assessment device having a closed body, such that the band is held within the body of the device by exertion of force by the expanded band against the inner surface of the device.

According to some embodiments, the band further comprises a wireless connection configured to provide operation and/or charging of the light source.

According to some embodiments, the at least one light source is configured to provide uniform illumination of an intraoral cavity of a subject.

In another aspect, provided herein is a method for dental assessment, comprising: providing a dental assessment device comprising at least a proximal opening configured for insertion within an intraoral region of a subject, and a distal opening: providing the band as disclosed herein: inserting the band within the dental assessment device, wherein the light source is directed towards the proximal end of the device: inserting the proximal end of the device within an intraoral region of a subject: illuminating at least one tooth within said intraoral region by the light source; and observing a dental fluorescence resulting from the illuminating of the at least one tooth.

According to some embodiments, inserting the band within the dental assessment device comprises inserting at a specific location in order to provide a required illumination.

According to some embodiments, the method further comprises: providing a camera: attaching the camera to the dental assessment device, wherein a lens of the camera is positioned to acquire an image or a video through the proximal opening via the distal opening, wherein observing the dental fluorescence comprises observing through the camera.

According to some embodiments, the attaching of the camera to the device is achieved using magnets, Velcro or stickers.

According to some embodiments, the method further comprises capturing and saving the image.

According to some embodiments, the camera is a camera of a mobile phone. According to some such embodiments, the camera and/or the mobile phone comprises software and/or hardware configured to analyze the acquired image and to identify a location and/or condition of at least a portion of at least one tooth of the subject. According to some such embodiments, the condition comprises a change (such as a reduction) in enamel and/or dentin.

In another aspect, provided herein is an intraoral element comprising: at least one light source providing illumination at a wavelength in the range of 360 nm to 440 nm: a power source configured to provide power to the light source: a switch configured to activate the light source; and at least one attachment component for reversibly attaching the light source to a location within an intraoral region of a subject.

According to some embodiments, the attachment component comprises a strip configured to be held between upper and lower teeth of a subject.

According to some embodiments, light is emitted from at least a portion of the light source. According to some embodiments, the portion comprises an entirety of the light source. According to some embodiments, the portion comprises less than an entirety of the light source. According to some such embodiments, the portion comprises 50% of the light source.

According to some embodiments, the light source provides uniform illumination of an intraoral region of a subject.

According to some embodiments, the switch is configured to be remotely activated.

According to some embodiments, the at least one light source comprises a first light source and at least a second light source, wherein the second light source is configured to provide light at a different wavelength range from that of the first light source, and wherein the first light source and the second light source are configured to be activated independently.

In another aspect, provided herein is a method for dental assessment, comprising: providing intraoral element with at least one light source providing illumination at a wavelength in a range of 360 nm to 440 nm: inserting the intraoral element into an intraoral region of a subject: illuminating an interior surface of at least one tooth within the intraoral region of the subject by the intraoral element; and observing a dental fluorescence resulting from the illuminating of the at least one tooth.

According to some embodiments, the method further comprises providing a dental assessment device comprising at least a proximal opening configured for insertion within an intraoral region of a subject; and a distal opening, wherein observing of the dental fluorescence is performed via the dental assessment device.

According to some embodiments, the method further comprises providing a camera: attaching the camera to the dental assessment device, wherein a lens of the camera is positioned so as to acquire an image or video through the proximal opening via the distal opening, wherein observing the dental fluorescence comprises observing through the camera.

According to some embodiments, the camera is a camera of a mobile phone.

According to some embodiments, the camera and/or the mobile phone comprises software and/or hardware configured to analyze an acquired dental image and to identify a location and/or condition of at least one tooth of the subject. According to some such embodiments, the condition comprises a change (such as a reduction) in enamel and/or dentin.

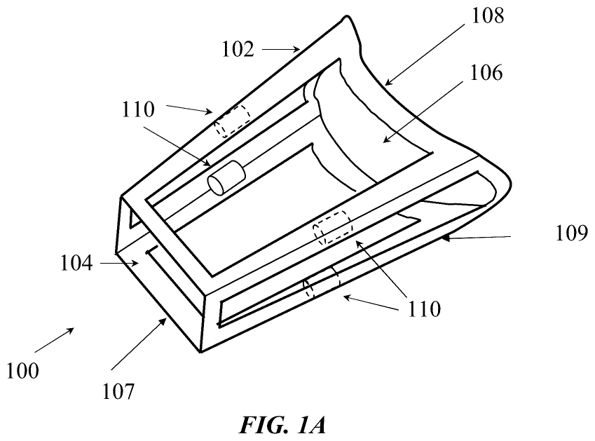

FIG. 1A schematically illustrates an embodiment of a dental assessment device 100 comprising an elongated body 102 having a distal opening 104: a proximal opening 106; and four light sources 110.

Body 102 can comprise an open-frame structure having a proximal edge 108, a distal edge 107 and four side sections 109 connecting between proximal edge 108 and distal edge 107. Body 102 can be configured to enable viewing of an image through proximal opening 106 via distal opening 104. A distance between proximal opening 106 and distal opening 104 can be at least 60 mm.

In some cases, distal edge 107 has a rectilinear shape.

Proximal opening 106 can have a suitable size and shape for insertion within an intraoral region of a subject and can have dimensions (length and width) at least equal to that of a surface (such as an upper surface or a front surface) of at least a single tooth.

Each one of the four light sources 110 can be positioned on a different one of the four side sections 109 and can be configured to emit light towards proximal opening 106, thereby providing uniform illumination at a wavelength in the range of 360 nm to 440 nm through proximal opening 106. Light sources 110 may be provided as light strips. In some cases, light sources 110 comprise light-emitting diodes (LEDs).

In some cases, the perimeter of proximal opening 106 is greater than that of distal opening 104. In alternative embodiments, the perimeter of proximal opening 106 may be substantially the same as that of distal opening 104.

FIG. 1B schematically illustrates an embodiment of an alternative dental assessment device 120 comprising a cylindrical body 122 having a distal opening 124: a proximal opening 126 having an outwardly-extending rim 128; and a plurality of light sources 130.

Body 122 can be configured to enable viewing of an image through proximal opening 126 via distal opening 124. In some cases, a distance between proximal opening 126 and distal opening 124 is at least 60 mm.

Body 122 can be opaque in order to block substantially all external illumination when inserted into an intraoral region of a subject.

Proximal opening 126 can have a suitable size and shape for insertion within an intraoral region of a subject and has dimensions (length and width) at least equal to that of a surface (such as an upper surface or a front surface) of at least a single tooth.

In some cases, each one of the plurality of light sources 130 is positioned at a different spaced-apart location within body 122 and is configured to emit light towards proximal opening 126, thereby providing uniform illumination at a wavelength in the range of 360 nm to 440 nm through proximal opening 126. Light sources 130 may be provided as light strips. In some cases, light sources 130 comprise light-emitting diodes (LEDs).

During use, when device 120 is inserted into an intraoral cavity of a subject, outwardly-extending rim 128 can support an inner surface of a lip of the subject, thereby separating the upper and lower lips from each other and/or from a surface of the teeth, thereby enabling unobstructed viewing of an image of at least one tooth.

FIG. 1C schematically illustrates a further alternative embodiment of a dental assessment device 140 comprising an elongated body 142 having a distal opening 144: a proximal opening 146; and a plurality of light sources 150.

Body 142 can be configured to enable viewing of an image through proximal opening 146 via distal opening 144. In some cases, a distance between proximal opening 146 and distal opening 144 is at least 60 mm.

Proximal opening 146 can have a suitable size and shape for insertion within an intraoral region of a subject and has dimensions (length and width) at least equal to that of a surface (such as an upper surface or a front surface) of at least a single tooth.

Body 142 can be opaque in order to block substantially all external illumination when inserted into an intraoral region of a subject.

In some cases, body 142 comprises a curved proximal edge 148, configured to fit against a human gingiva, a distal edge 147 and four sides 149 between proximal edge 148 and distal edge 147.

Each one of the plurality of light sources 150 can be positioned at a different spaced-apart location within body 142 and is configured to emit light towards proximal opening 146, thereby providing uniform illumination at a wavelength in the range of 360 nm to 440 nm through proximal opening 146. Light sources 150 may be provided as light strips. In some cases, light sources 150 comprise light-emitting diodes (LEDs).

In some cases, the perimeter of proximal opening 146 is greater than that of distal opening 144. In alternative embodiments, the perimeter of proximal opening 146 may be substantially the same as that of distal opening 144.

According to any of the embodiments disclosed herein, the dental assessment device can be formed from any suitable biocompatible material having a desired level of flexibility/rigidity to maintain the shape of the device when a proximal opening is inserted into an intraoral region of a subject and to support multiple light sources without deformation and is suitable for cleaning after use. The dental assessment device can be suitable for multiple uses by a single subject.

According to any of the embodiments of the dental assessment device disclosed herein, a body of the device may be configured to enable viewing of an image through a camera, when a lens of the camera is positioned so as to acquire an image through a proximal opening via a distal opening of the device. In some embodiments, the camera is a digital camera, such as a digital camera of a mobile phone or a stand-alone digital camera. In some embodiments, a reversibly attachable digital camera is provided as a component of a system comprising the dental assessment device.

According to some embodiments, a dental assessment device as described herein can have a mounting mechanism for coupling a camera or a mobile device to a body of the dental assessment device. The mounting mechanism may be configured to couple the camera or mobile device to the body of the dental assessment device such that a longitudinal axis of a viewing channel between the distal opening and the proximal opening of the device is substantially aligned with an optical axis of one or more cameras of the mobile device. The mounting mechanism may be configured to mechanically engage with the mobile device or a casing of the mobile device. In some embodiments, the mounting mechanism may comprise an elastic band, a clamp, a hook, a magnet, a bracket, or a holder.

The optical axis of the one or more cameras of the mobile device may be aligned with one or more intraoral regions of the subject's mouth when a flange of the elongated housing is positioned between a tooth portion and a gum portion of the subject's mouth. The mobile device may comprise an imaging device (e.g., a camera) that can be configured to capture the one or more intraoral images or videos.

The viewing channel of the elongated housing may be configured to define a field of view of an intraoral region of a subject's mouth. The field of view may be sized and/or shaped to permit one or more cameras of the mobile device to capture one or more videos or images of one or more intraoral regions in a subject's mouth. In some cases, the videos may comprise one or more intraoral images showing a full dental arch of the subject.

According to any of the embodiments disclosed herein, a proximal end of dental assessment device having an outwardly extending rim may be sized and shaped to couple the dental assessment device to the subject's mouth when the rim is positioned between a gum portion and a tooth portion of the subject's mouth. The dental assessment device may be suspended from the subject's mouth when the rim is positioned between the gum portion and the tooth portion of the subject's mouth. The gum portion and the tooth portion may be in contact with a first side of the rim and a second side of the rim to support a weight of the dental assessment device when the dental assessment device is suspended from the subject's mouth. The rim may be sized and shaped to permit the subject to move the dental assessment device and/or to adjust a position or an orientation of the dental assessment device relative to one or more intraoral regions in the subject's mouth.

In dental assessment devices comprising a mobile device having a camera, adjusting the position or the orientation of the dental assessment device relative to one or more intraoral regions in the subject's mouth may also adjust a position or an orientation of the camera of the mobile device relative to the one or more intraoral regions in the subject's mouth. Adjusting a position or an orientation of the camera of the mobile device relative to the one or more intraoral regions in the subject's mouth may further adjust a relative position and/or a relative orientation of an optical axis of the camera relative to the one or more intraoral regions in the subject's mouth. The rim remains between the gum portion and the tooth portion of the subject's mouth while the subject moves the dental assessment device around in the subject's mouth. The rim may be sized and shaped to permit the subject to capture one or more intraoral videos or images of a full dental arch of the subject. In any of the embodiments described herein, the rim of the dental assessment device may be configured to be positioned outside the field of view defined by the viewing channel of the dental assessment device.

According to some embodiments, the camera comprises an electronic storage device, such as a digital memory card, to save and store an acquired image.

The camera may be used to capture intraoral videos or images. In some cases, the dental assessment device may comprise an attachment mechanism for coupling a mobile device to the body of the dental assessment device. The attachment mechanism may comprise, for example, a strap for securing the mobile device to the body of the dental assessment device. The strap may comprise a flexible and/or compliant material, such as silicone. In some cases, the strap may comprise any biocompatible material, or any material that is dishwasher safe. The strap may be adjustable to enable a user to couple various mobile devices having different sizes, shapes, and/or form factors. The adjustability of the strap may provide several advantages, including improved compatibility with different mobile devices having distinct camera configurations, or imaging sensors disposed on different portions or locations on the mobile device.

The method may permit the patient to take one or more intraoral images or videos. The intraoral images or videos may be capture while the patient is moving the intraoral adapter, or after the patient moves the intraoral adapter to a predetermined location.

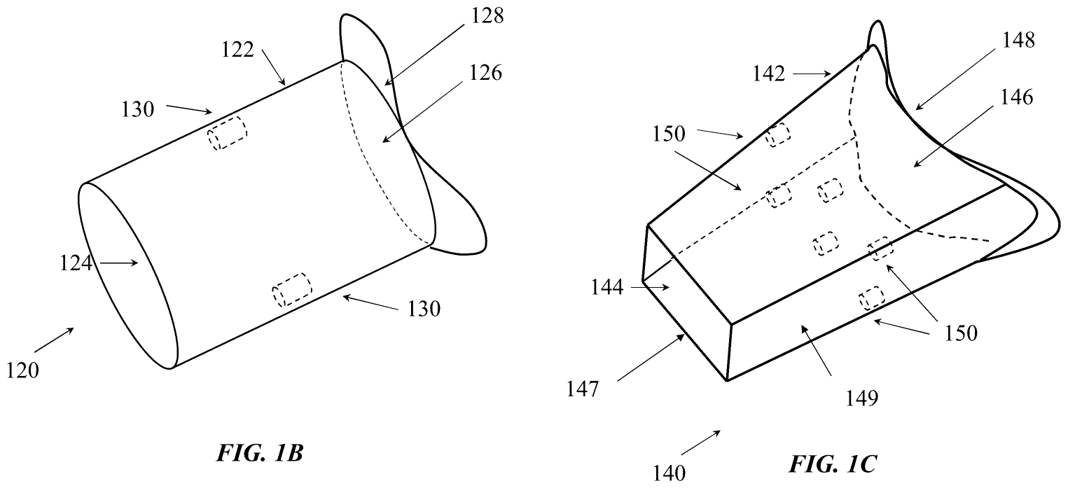

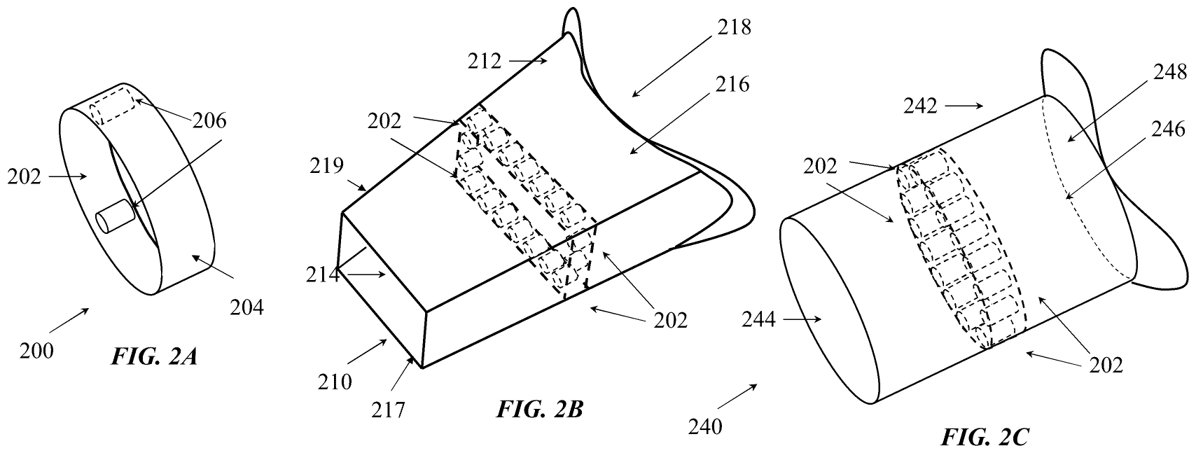

FIG. 2A schematically illustrates an example of a band 200, in accordance with some embodiments. Band 200 can have an inner face 202, an outer face 204, and two edges; and at least two light sources 206 proving illumination at a wavelength in the range of 360 nm to 440 nm, positioned at spaced-apart locations on band 200. Light sources 206 can be positioned on band 200 so as to provide illumination in a direction of one edge of band 200.

Band 200 can be a self-expanding band. i.e. a band which can be provided in a compact form when restrained, which expands upon removal of a restraint.

In some cases, band 200 further comprises a power source configured to provide power to each light source 206 and a switch configured to activate light source 206. According to some embodiments, the switch is a remotely controlled switch.

FIG. 2B schematically illustrates an example of a band 200 positioned within a dental assessment device 210, wherein dental assessment device 210 comprises a body 212 having a distal opening 214; and a proximal opening 216.

In some cases, body 212 comprises a curved proximal edge 218, a distal edge 217 and four sides 219 extending between proximal edge 218 and distal edge 217.

Upon insertion of band 220 into body 212, band 220 can extend so as to exert pressure against an inner surface of each one of sides 219 thereby retaining the position of band 220 within body 212. In some cases, band 220 is positioned such that light sources 206 are oriented so as to emit light towards proximal opening 216, thereby providing uniform illumination at a wavelength in the range of 360 nm to 440 nm through proximal opening 216

The perimeter of proximal opening 216 can be greater than that of distal opening 214. In alternative embodiments, the perimeter of proximal opening 216 may be substantially the same as that of distal opening 214.

FIG. 2C schematically illustrates an example of a band 200 positioned within a dental assessment device 240, wherein dental assessment device 240 comprises a cylindrical body 242 having a distal opening 244; and a proximal opening 246 having an outwardly-extending rim 248.

Upon insertion of band 220 into body 212, band 220 may extend so as to exert pressure against an inner surface of body 242, thereby retaining the position of band 220 within body 242. Band 220 can be positioned such that lights 206 are oriented so as to emit light towards proximal opening 246, thereby providing uniform illumination at a wavelength in the range of 360 nm to 440 nm through proximal opening 246.

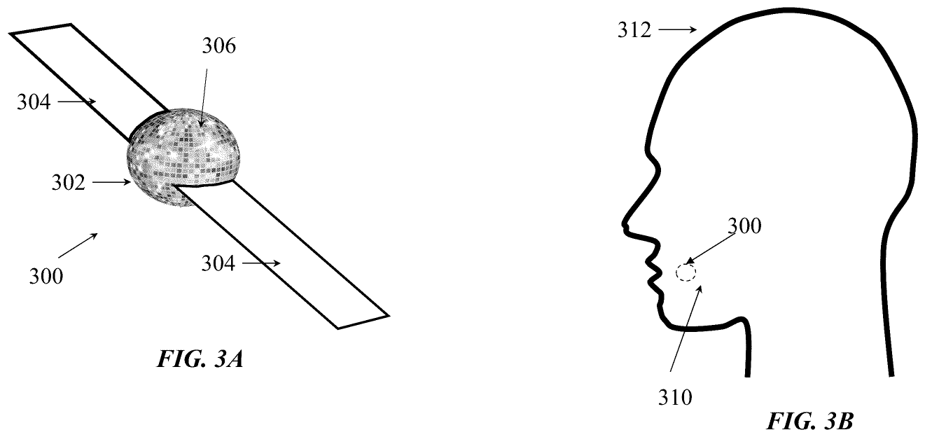

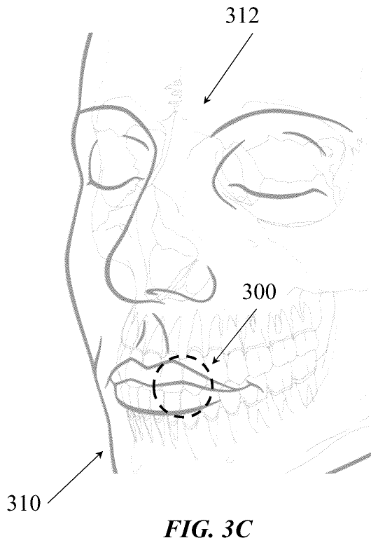

FIG. 3A schematically illustrates an example of an intraoral element 300 comprising a light source 302 configured to provide illumination at a wavelength in the range of 360 nm to 440 nm; and a strip 304 consisting of two flanges extending outwardly from opposite sides of light source 302. According to an embodiment, as shown in FIG. 3A, light source 302 can be provided in the form of a sphere 306. According to alternative embodiments (not shown), the light source of intraoral element 300 may be provided in any suitable shape, such as a rectangle, a cube or the like.

According to some embodiments, light is emitted from at least a portion of light source 302, such as from an entirety of an outer surface of light source 302 or from a portion which is less than an entirety of the outer surface. For example, light may be emitted from a region in the shape of a strip around the circumference, or around part of the circumference, of a spherical light source 302.

According to some embodiments (not shown), an intraoral element may comprise at least two light sources wherein a second light source is configured to provide light at a different wavelength range from that of a first light source, and wherein the first light source and the second light source are configured to be activated independently.

Strip 302 of intraoral element 300 may serve to provide reversible or releasable attachment of light source 302 within an intraoral cavity of a subject.

Intraoral element 300 may further comprises a power source configured to provide power to each light source 302 and a switch configured to activate light source 302. According to some embodiments, the switch is a remotely controlled switch.

During use, as shown schematically in FIGS. 3B and 3C, an intraoral element 300 according to FIG. 3A, can be inserted within an intraoral cavity 310 of a subject 312. Light source 302 can be positioned on the tongue of subject 312 with the flanges of strip 304 extending over the lower teeth substantially parallel to the front of the face of subject 312. In some cases, teeth of subject 312 are then closed such that strip 304 holding light source 302 are held between upper and lower side teeth of subject 312, such that light source 302 is held in a fixed position on the tongue of subject 312, wherein light source 302 provides illumination within the intraoral region of subject 312 and back illumination to the teeth.

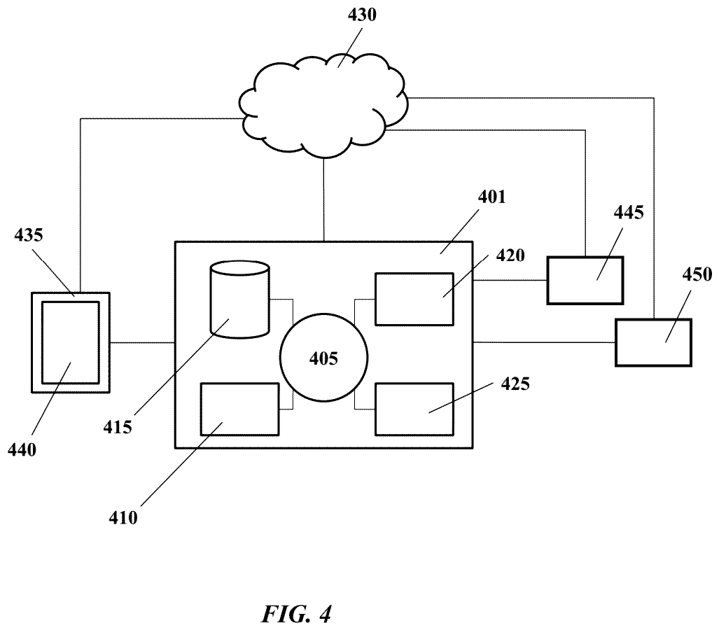

FIG. 4 schematically illustrates a computer system that is programmed or otherwise configured to implement at least some of the methods disclosed herein, in accordance with some embodiments.

### Computer Systems

In an aspect, the present disclosure provides computer systems that are programmed or otherwise configured to implement methods of the disclosure. FIG. 4 shows a computer system 401 that is programmed or otherwise configured to implement a method for dental assessment. The computer system 401 may be configured to, for example, process intraoral videos or images captured using the camera of the mobile device, and determine at least one of the dental assessment properties during the dental assessment. The computer system 401 may be configured to, for example, process orientation or motion sensors data using the orientation or motion sensors of the mobile device, and determine at least one of the dental assessment properties during the dental assessment. The computer system 401 may be configured to activate a feedback element to generate output. The computer system 401 can be an electronic device of a user or a computer system that is remotely located with respect to the electronic device. The electronic device can be a mobile electronic device. The computer system 401 can be a smartphone.

The computer system 401 may include a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk, Solid State drive or equivalent storage unit), communication interface 420) (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are located external to the computer system 401 (e.g., on a remote server that is in communication with the computer system 401 through an intranet or the Internet).

The computer system 401 can communicate with one or more remote computer systems through the network 430. For instance, the computer system 401 can communicate with a remote computer system of a user (e.g., a subject, a dental patient, or a dentist). Examples of remote computer systems include personal computers (e.g., portable PC), slate or tablet PC's (e.g., Apple® iPad, Samsung R; Galaxy Tab), telephones, Smart phones (e.g., Apple® iphone, Android-enabled device, Blackberry R), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g. read-only memory, random-access memory, flash memory) or a storage unit. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables: copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM. DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, a portal for a subject or a dental patient to view one or more intraoral images or videos captured using a mobile device of the subject or the dental patient. In some cases, the electronic display 435 may be the feedback element providing the generated output, for example displaying message or shape or light in accordance to some embodiments. The portal may be provided through an application programming interface (API). A user or entity can also interact with various elements in the portal via the UI. Examples of UI's include, without limitation, a graphical user interface (GUI) and web-based user interface.

The computer system 401 can include or be in communication with a Camera 445 for providing, for example, ability to capture videos or images of the subject or a dental patient. And for example, retrieve at least one dental scan date (such as optical object distance) that can be used to analyzed and compered to at least one dental scan properties

Methods and systems of the present disclosure can be implemented by way of one or more algorithms. An algorithm can be implemented by way of software upon execution by the central processing unit 405. The algorithm can, for example, implement a method for dental scan. The method may comprise processing videos or images captured using the camera of the mobile device or processing dental assessment data sensed by at least one sensor that can be used to analyze and compare to at least one dental assessment properties and executed to generate output.

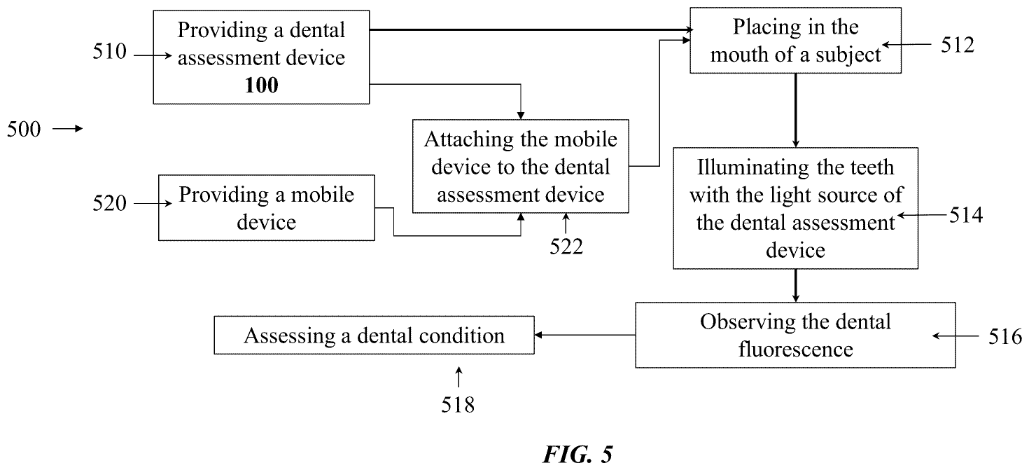

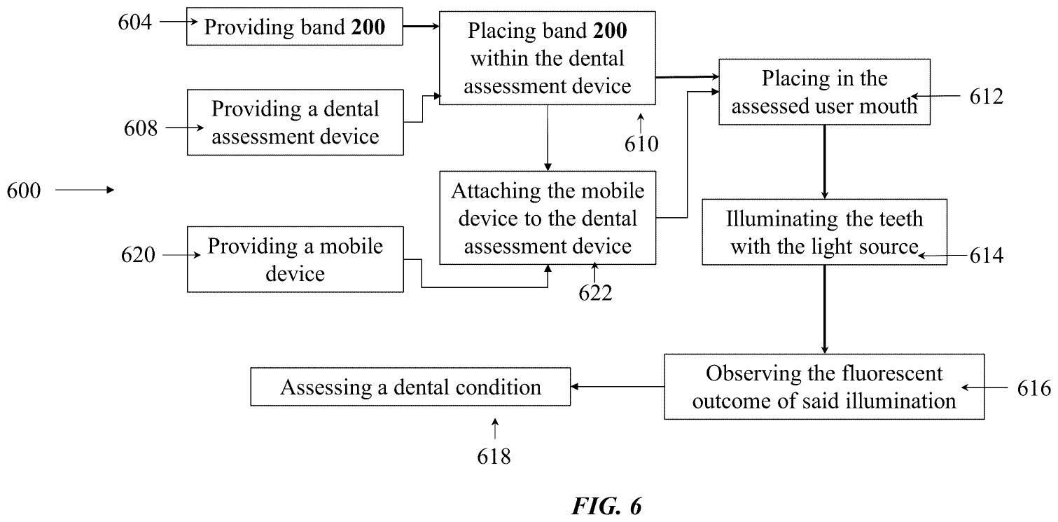

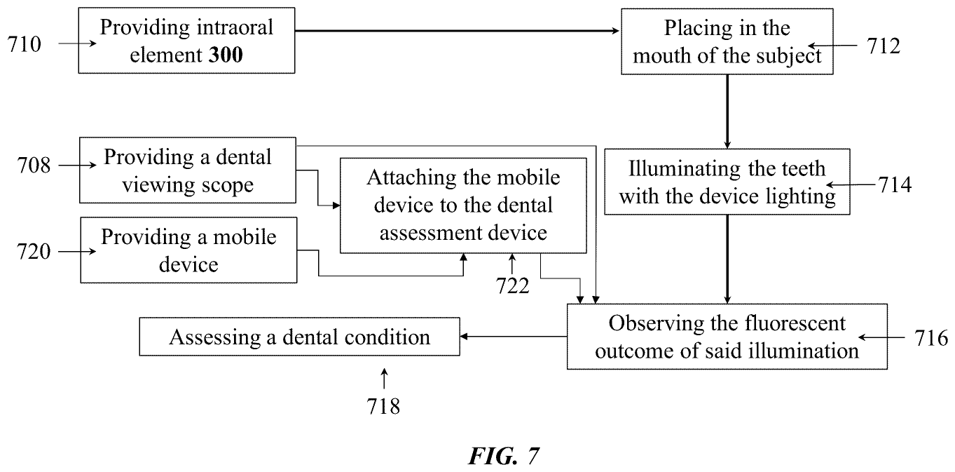

FIG. 5. FIG. 6, and FIG. 7 are flow-charts schematically illustrating methods for dental assessment, in accordance with some embodiments.

FIG. 5 illustrates an example of a method 500 for dental assessment using a dental assessment device as disclosed herein (such as device 100, 120 or 140). For the sake of clarity, reference is made herein to device 100.

Dental assessment device 100 and a mobile device can be configured to acquire an image or a video are provided to a subject (steps 510, 520, respectively). The subject may attach the mobile device to dental assessment device 100 (step 522) such that a camera within the mobile device is positioned to enable viewing through proximal opening 106 via distal opening 104. Proximal end 106 of device 100 can then be inserted into the mouth of the subject (step 512) and light source 110 activated to as to illuminate the teeth of the subject (step 514). The resulting dental fluorescence may be observed by a user (516), optionally by a dental professional, optionally at a remote location. In some cases, a dental condition is assessed (step 518) according to the observed dental fluorescence.

According to an alternative method (not shown) end 106 of device 100 may be inserted into the mouth of the subject (step 512) and light source 110 activated to as to illuminate the teeth of the subject (step 514). The resulting dental fluorescence can be observed by a user (516). In some cases, a dental condition is assessed (step 518) according to the observed dental fluorescence.

FIG. 6 illustrates an example of a method 600 for dental assessment using a dental assessment device comprising a proximal opening configured for insertion within an intraoral region of a subject and a distal opening; and a band 200 as disclosed herein.

Band 200, the dental assessment device and a mobile device can be provided to a subject (steps 604, 608, 620, respectively). The subject may insert band 200 within dental assessment device (step 610) and attach the mobile device to the dental assessment device (step 622) such that a camera within the mobile device is positioned to enable viewing of an image through the proximal opening via the distal opening of the dental assessment device. The proximal end of the dental assessment device can be inserted into the mouth of the subject (step 612) and the light source of band 200 can be activated so as to illuminate the teeth of the subject (step 614). The resulting dental fluorescence can be observed by a user (step 616), optionally by a dental professional, optionally at a remote location. In some cases, a dental condition is assessed (step 618) according to the observed dental fluorescence.

According to an alternative method (not shown), band 200 and the dental assessment device are provided to a subject (steps 604, 608, respectively). The subject may insert band 200 within dental assessment device (step 610) The proximal end of the dental assessment device can be inserted into the mouth of the subject (step 612) and the light source of band 200 can be activated to as to illuminate the teeth of the subject (step 614). In some cases, the resulting dental fluorescence is observed by a user (step 616), and a dental condition is assessed (step 618) according to the observed dental fluorescence.

FIG. 7 illustrates an example of a method 700 for dental assessment using a dental assessment device comprising a proximal opening configured for insertion within an intraoral region of a subject and a distal opening; and an intraoral element 300 as disclosed herein.

Intraoral element 300, the dental assessment device and a mobile device can be provided to a subject (steps 710, 708, 720, respectively).

The mobile device can be attached to the dental assessment device (step 722) such that a camera within the mobile device is positioned to enable viewing of an image through the proximal opening via the distal opening of the dental assessment device.

Intraoral element 300 can then be placed into the mouth of the subject (step 712) and the light source 302 is of intraoral element 300 can be activated to illuminate the teeth of the subject (step 714).

In some cases, the proximal end of the dental assessment device is inserted into the mouth of the subject and the resulting dental fluorescence is observed by a user (step 716), optionally by a dental professional, optionally at a remote location. In some cases, a dental condition is assessed (step 718) according to the observed dental fluorescence.

The dental scan disclosed herein may be used with any type of dental assessment device that is configured to permit capture of a patient's teeth or dental structure. The dental assessment device may be configured to permit the patient to capture one or more intraoral videos or images using a mobile device or a smartphone. The intraoral adapter may be configured to position the mobile device or smartphone such that the patient is able to capture the images or videos from one or more predetermined positions or viewing angles.

While certain embodiments have been shown and described herein, it will be obvious to those skilled in the art that such embodiments are provided by way of example only. It is not intended that the systems and methods described herein be limited by the specific examples provided within the specification. While the systems and methods described herein have been described with reference to the aforementioned specification, the descriptions and illustrations of the embodiments herein are not meant to be construed in a limiting sense.

Numerous variations, changes, and substitutions will now occur to those skilled in the art without departing from the systems and methods described herein. Furthermore, it shall be understood that all aspects of the systems and methods described herein are not limited to the specific depictions, configurations or relative proportions set forth herein which depend upon a variety of conditions and variables. It should be understood that various alternatives to the embodiments described herein may be employed in practicing the systems and methods described herein. It is therefore contemplated that the systems and methods described herein shall also cover any such alternatives, modifications, variations or equivalents. It is intended that the following claims define the scope of the systems and methods described herein and that methods and structures within the scope of these claims and their equivalents be covered thereby.

1. A dental assessment device, comprising:
a body comprising
(i) a proximal opening, wherein said proximal opening is configured to be inserted within an intraoral region of a subject, and wherein said proximal opening has dimensions at least equal to that of a surface of a single tooth of said subject;
(ii) a distal opening, wherein said body is configured to enable viewing through said proximal opening via said distal opening, and wherein a distance between said proximal opening and said distal opening is at least 60 mm; and
(iii) at least one light source configured to provide uniform illumination through said proximal opening at a wavelength in a range of 360 nm to 440 nm.

2. The dental assessment device of claim 1, wherein said body is configured to enable viewing through said proximal opening through a camera, and wherein a lens of said camera is positioned to acquire an image or a video through said proximal opening via said distal opening.

3. The dental assessment device of claim 1, wherein said proximal opening is configured to fit against a human gingiva.

4. The dental assessment device of claim 1, wherein said proximal opening is curved.

5. The dental assessment device of claim 1, wherein said proximal end of the body is configured to couple the body to the subject's mouth.

6. The dental assessment device of claim 5, wherein said proximal end comprises alternating concave and convex regions.

7. The dental assessment device of claim 6, wherein said concave regions are configured to contact a top and bottom lip of said subject's mouth.

8. The dental assessment device of claim 7, wherein said concave regions are configured to hold a lip of said subject outside of said proximal end.

9. The dental assessment device of claim 6, wherein said convex regions are configured to contact a side of said subject's mouth.

10. The dental assessment device of claim 9, wherein said convex regions are configured to hold a cheek of said subject outside of said proximal end.

11. The dental assessment device of claim 1, further comprising an outwardly extending rim around said proximal opening.

12. The dental assessment device of claim 1, wherein said proximal opening is configured to support an inner surface of a lip of a subject when inserted into an intraoral region of said subject proximal to a gingiva.

13. The dental assessment device of claim 1, wherein said body is configured to block substantially all external illumination when inserted into an intraoral region of a subject proximal to a gingiva of said subject.

14. A method for dental assessment, comprising:
(a) providing the device of claim 1:
(b) inserting said proximal opening into an intraoral region of a subject;
(c) illuminating at least one tooth within said intraoral region of said subject with said at least one light source; and
(d) observing a dental fluorescence resulting from said illuminating of said at least one tooth.

15. The method for dental assessment of claim 14, wherein the method further comprises:
(a) providing a camera; and
(b) attaching said camera to said device, wherein a lens of said camera is positioned to acquire an image or a video through said proximal opening via said distal opening, wherein said camera is used for said observing of said dental fluorescence.

16. A band for dental assessment, comprising:
(a) an inner face;
(b) an outer face;
(c) two edges;
(d) at least one light source configured to provide illumination at a wavelength in the range of 360 nm to 440 nm, wherein said illumination extends outwardly from at least one of said two edges;
(e) a power source configured to provide power to said light source; and
(f) a switch configured to activate said light source.

17. A method for dental assessment, comprising:
(a) providing a dental assessment device comprising (i) a proximal opening configured for insertion within an intraoral region of a subject, and (ii) a distal opening:
(b) providing said band of claim 16;
(c) inserting said band in said dental assessment device, wherein said light source is directed towards said proximal opening of said device;
(d) inserting said proximal opening of said device in an intraoral region of a subject;
(e) illuminating at least one tooth of said subject within said intraoral region with said light source; and
(f) observing a dental fluorescence resulting from said illuminating of said at least one tooth of said subject.

18. An intraoral element comprising:
(a) at least one light source,
(b) a power source configured to provide power to said light source;
(c) a switch configured to activate said light source; and
(d) at least one attachment component for releasably coupling said light source to a location within an intraoral region of a subject.

19. The intraoral element of claim 18, wherein said light source is configured to provide illumination at a wavelength in the range of 360 nm to 440 nm.

20. A method for dental assessment, comprising;
(a) providing said intraoral element of claim 19;
(b) inserting said intraoral element into an intraoral region of a subject;
(c) using said intraoral element, illuminating an interior surface of at least one tooth of said subject within said intraoral region; and
(d) observing a dental fluorescence resulting from said illuminating of said at least one tooth of said subject.
